(19) United States
(12) Patent Application Publication
(10) Pub. No.: US 2026/0215754 A1
(43) Pub. Date: Jul. 30, 2026

(54) SYSTEM AND METHOD FOR ESTIMATING THE METASTATIC STATUS OF A SENTINEL LYMPH NODE

(71) Applicant: I.R.C.C.S. ISTITUTO TUMORI “GIOVANNI PAOLO II”, BARI (IT)

(72) Inventors: Raffaella MASSAFRA, BARI (IT); Annarita FANIZZI, BARI (IT); Maria Colomba Comes, BARI (IT); Samantha BOVE, BARI (IT)

(21) Appl. No.: 19/145,321

(22) PCT Filed: Jan. 4, 2024
(86) PCT No.: PCT/IT2024/050001

§ 371 Date: Jul. 2, 2025

(30) Foreign Application Priority Data
Jan. 5, 2023 (IT) 102023000000105

(51) Int. Cl. A61B 8/08; A61B 8/00; G06T 7/00; G06T 7/12; G06V 10/25; G06V 10/26; G06V 10/44; G06V 10/764; G06V 10/774; G06V 10/82; G16H 30/40; G16H 50/20

(52) U.S. Cl. CPC A61B 8/085; A61B 8/469; A61B 8/5223; G06T 7/0012; G06T 7/12; G16H 30/40; G16H 50/20; G06T 2207/10132; G06T 2207/20076; G06T 2207/20081; G06T 2207/20084; G06T 2207/30096; G06T 2207/30204; G06V 10/25; G06V 10/26; G06V 10/44; G06V 10/764; G06V 10/774; G06V 10/82; G06V 2201/03

(57) ABSTRACT

A computer-implemented method for estimating the metastatic status of a sentinel lymph node. The method comprises: acquiring an ultrasound image of the primary tumor; segmenting said ultrasound image, wherein segmenting said ultrasound image comprises determining a region of interest, ROI, comprising an intratumoral region of the primary tumor and a peritumoral region of the primary tumor and wherein segmenting said ultrasound image comprises performing a semantic segmentation process using a CNN that was trained to determine said intratumoral region of the primary tumor; extracting a plurality of characteristics of said image from said ROI using a pre-trained convolutional neural network, CNN; selecting one or more characteristics of said plurality of characteristics; and classifying the metastatic status of said sentinel lymph node based on said one or more characteristics.

The present invention relates to a system and a method for classifying metastatic status of a sentinel lymph node (SLN).

In particular, the present invention relates to a computer-implemented method that allows to classify the metastatic status of a sentinel lymph node in patients affected by breast cancer and with clinically negative lymph nodes, by analyzing exclusively the characteristics of the primary tumor.

This invention also relates to a computer program product and a system configured to classify the metastatic status of a sentinel lymph node.

### BACKGROUND OF THE INVENTION

An accurate detection of the metastatic status is essential to reduce the chance of future occurrence of metastasis in patients affected by breast carcinoma.

After a first breast cancer diagnosis, if the patient has been found negative both for clinical and instrumental examination, the lymph status is commonly evaluated by performing an intraoperative procedure.

The intraoperative procedure involves biopsy of the sentinel lymph node, which is the first lymph node to be reached from a primary tumor as it is proximal to the primary tumor. The intraoperative procedure involves the use of a molecular method for the identification of metastatic cells inside the lymph nodes, such as the OSNA (One Step Nucleic Acid Amplification) method. Such intraoperative procedure is invasive and is expensive both in terms of time and in terms of costs. In addition, this procedure can induce significant side effects.

Bove, S., Comes, M. C., Fanizzi, A. & Massafra, R. “A ultrasound-based radiomic approach to predict the nodal status in clinically negative breast cancer patients” Sci Rep 12, 7914 (2022). https://doi.org/10.1038/s41598-022-11876-4 (12 May 2022) proposes a non-invasive alternative to the OSNA procedure described above.

This non-invasive procedure makes it possible to estimate the status of the sentinel lymph node by means of a machine learning model capable of predicting the lymph status from an ultrasound image of the primary tumor.

In such a procedure, the ultrasound image of the acquired primary tumor is processed in order to obtain four different regions of interest (original ROI, intratumoral ROI, peritumoral ROI, combined ROI) previously manually identified by the radiologist, then radiomic features are extracted, i.e. a limited number of features manually extracted by the operator that describe both the tissue characteristics, but also quantitative and exemplary characteristics of the distribution of gray levels within the image.

The determination of a status of the sentinel lymph node is based on the joint analyses of the clinical variables with the radiomic features extracted from the four different regions of interest. These clinical variables and radiomic features are correlated with the metastatic nodal status. By means of this joint analysis, it is possible to determine whether the primary tumor has acquired the ability to migrate to the sentinel lymph node, or that has migrated, thus obtaining an estimate of the metastatic status of the sentinel lymph node.

Although the joint analysis of clinical variables with radiological characteristics extracted from the ROI represents an alternative applicable to clinical practice, this method is based on the operator's experience and it would therefore be beneficial to optimize such a method in order to improve accuracy and applicability to clinical practice.

### OBJECT OF THE INVENTION

The aim of the present invention is to overcome said disadvantages, by providing a method to classify the metastatic status of a sentinel lymph node.

Therefore, the object of the invention is a method as claimed in claim 1, a system as claimed in claim 12 and a computer program product as claimed in claim 13.

Further preferred embodiments are described in the dependent claims.

### BRIEF DESCRIPTION OF THE DRAWINGS

The present invention will now be described, for illustrative but not limitative purposes, according to an embodiment thereof, with particular reference to the accompanying figures, in which:

FIG. 1 shows a block diagram of a method for classifying the status of a sentinel lymph node, according to an embodiment of the present invention;

FIG. 2a shows an ultrasound image of the primary tumor comprising one or more markers, according to an embodiment of the present invention;

FIG. 2b shows the ultrasound image in FIG. 1 wherein said one or more markers have been selected, according to an embodiment of the present invention;

FIG. 2c shows the ultrasound image of FIG. 1 wherein said one or more markers have been removed and the pixels of the image have been reconstructed, according to an embodiment of the present invention;

FIG. 3 shows a binary mask wherein pixels other than zero (i.e. the pixels in white) correspond to the designated region of the primary tumor; and

FIG. 4 shows a region of interest of the ultrasound image that includes both the intratumoral region and the peritumoral region of the primary tumor.

### DETAILED DESCRIPTION OF THE INVENTION

In the various figures, similar parts will be indicated by the same reference numerals.

Certain steps of the method will be described below using specific instructions of the programming platform Matlab® but it is well evident that the invention should not be considered limited to the use of such a specific platform and/or instructions.

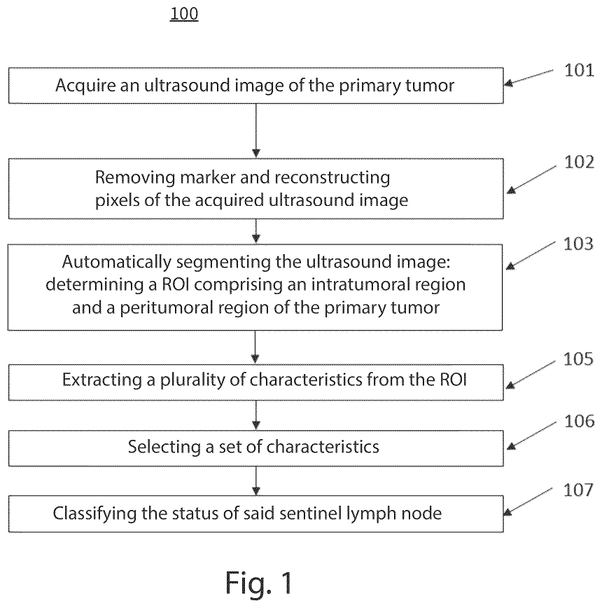

With reference to FIG. 1, a computer-implemented method 100 is shown for classifying the status of a sentinel lymph node, such as, for example, an axillary sentinel lymph node.

The method 100 makes it possible to predict, in an automatic manner, the status of the sentinel lymph node in patients affected by a breast cancer that is judged as being clinically negative, for example during the diagnostic step of the primary tumor, and on the basis of the use of an ultrasound image 1 of the primary tumor 10 that can be appropriately selected by the radiologist.

The method 100 comprises the step of acquiring 101 an ultrasound image 1 of the primary tumor 10, for example an image of a breast cancer.

The ultrasound image 1 can be obtained from an ultrasound examination by means of ultrasounds.

The image can be acquired directly by the system or it can be appropriately selected by the radiologist before being acquired by the system.

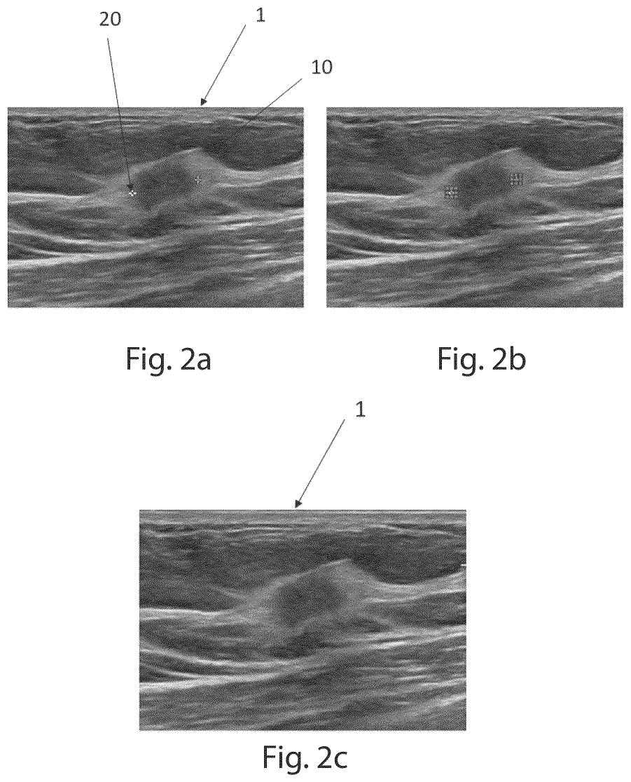

The ultrasound image 1 showing the primary tumor diagnosed by the radiologist acquired in step 101 may contain some markers 20 as shown for example in FIG. 2a.

In particular, during the screening step and after having identified the primary tumor 10, the radiologist can position some markers 20 on the ultrasound image 1 to delimit the region of interest containing the primary tumor 10. Therefore, the pixels of the original image corresponding to these markers 20 can be compromised.

The method 100 may comprise a step in which it is determined whether said ultrasound image 1 comprises one or more markers 20, wherein each of said one or more markers 20 is associated with one or more pixels of said ultrasound image 1.

The method 100 may further comprise a step in which each of said one or more markers 20 is removed and the one or more corresponding pixels are restored 102 before proceeding with the segmentation step 103 of the ultrasound image 1.

This step allows to remove the compromised pixels and to replace them by using, for example, an exemplar-based synthesis of the image, i.e., by generating a new image which is perceptually similar to the ultrasound image 1 obtained from the ultrasound examination.

In more details, the step of removing and restoring the pixels of the image may comprise the steps of:

A1. Associating the indicator I to the numerical array that represents the ultrasound image 1, verifying whether I represents a numerical array of size m-by-n-by-3, thus a Truecolor image, or a numerical array m-by-n that encodes a grayscale image.

If the image is a Truecolor image, it can be converted to grayscale. The conversion operation may, for example, be performed by using the Matlab® function rgb2gray that executes, pixels by pixel, a weighted sum of the RGB components as follows:

$$0.2989 * R + 0.5870 * G + 0.1140 * B .$$

A2. Obtaining the image J showing the marker 20 and detecting the marker 20 on the ultrasound image 1. For this purpose, the following procedure is implemented:
i. calculating the normalized cross-correlation in the spatial domain between the two matrices of the input image I and of the image J, using the Matlab® normxcorr2: this operation makes it possible to identify geographically related pixels in the two images.

ii. finding the coordinates (xpeak1, ypeak1) and (xpeak2, ypeak2) of the two peaks of the cross-correlation matrix calculated in step (i): the two peaks correspond to the final points of two markers 20 in I.

iii. identifying the final points of the other two markers 20 by adding the two dimensions of J to the coordinates (xpeak1, ypeak1) and (xpeak2, ypeak2).

iv. drawing a rectangle delimiting the corresponding area (FIG. 2b) using the four final points calculated in steps (ii) and (iii).

v performing the inpaint process utilizing the Matlab® inpaintCoherent function. Starting from the boundary pixels of the identified rectangle, generating a binary mask of the same size as the input image I where the pixels other than zero corresponds to the region to be designed. Thus, for each pixel in this region, computing the inpainted value as a weighted average of the known pixel values within the radius of inpainting. The weighted averages are assigned as follows: after estimating the coherence direction d, the pixels along d are associated with a higher average value than the pixels along other directions. Thus, the compromised pixel values are replaced with the corresponding calculated average values, obtaining an ultrasound image 1 without markers as shown, for example, in FIG. 2c.

With reference to FIG. 1, the method 100 comprises the segmentation step 103 of the ultrasound image. The segmentation stage 103 enables the region of interest (ROI) to be correctly identified, thus avoiding false candidates.

Step 103 comprises determining a ROI comprising an intratumoral region 12 of the primary tumor 10 and a peritumoral region 14 of the primary tumor 10 as shown, for example, in FIG. 4.

The segmentation step 103 includes performing a semantic segmentation process using a CNN that was trained to determine said intratumoral region 12 of the primary tumor 10.

The ROI determination step may further include a step of delineating the intratumoral region 12 of the primary tumor 10 and the peritumoral region 14 of the primary tumor 10.

As described below, the semantic segmentation process may utilize a Xception architecture such as a CNN pre-trained. The training may include a learning step using over three thousand ultrasound images acquired, for example, by seven ultrasonic devices. Said segmenting process is described in Wilfrido Gómez-Flores, Wagner Coelho de Albuquerque Pereira, “A comparative study of pre-trained convolutional neural networks for semantic segmentation of breast tumors in ultrasound”, Computers in Biology and Medicine, Volume 126, 2020, 104036, ISSN 0010-4825, (https://www.sciencedirect.com/science/article/pii/S001048252030367X).

The segmentation step 103 described above may comprise the following sub-steps:

B1. Associating the indicator I* to the numerical array that represents the ultrasound image 1 and resizing the ultrasound image 1 to obtain an image of dimensions 128×128 pixels, as required by the CNNXception architecture used.

B2. Implement a semantic segmentation procedure utilizing Matlab® semanticseg function. This function allows to classify each pixel in I*, assigning the label tumor/normal to each pixel. For each pixel, the assigned label can be subsequently allocated in a semantic array S of the same size as the input ultrasound image 1 of input I*.

B3. Resizing both the image I* and the semantic array S to the original dimension of the ultrasound image 1.

B4. Delineating both the identified intratumoral region 12 and a peritumoral region 14 which could store information relevant for the estimation of the status of the sentinel lymph node, since it comprises the connecting tissue between the primary tumor 10 and normal tissue, and thus represents an area of tumor proliferation and angiogenesis.

The delineation step can comprise the following sub-steps:

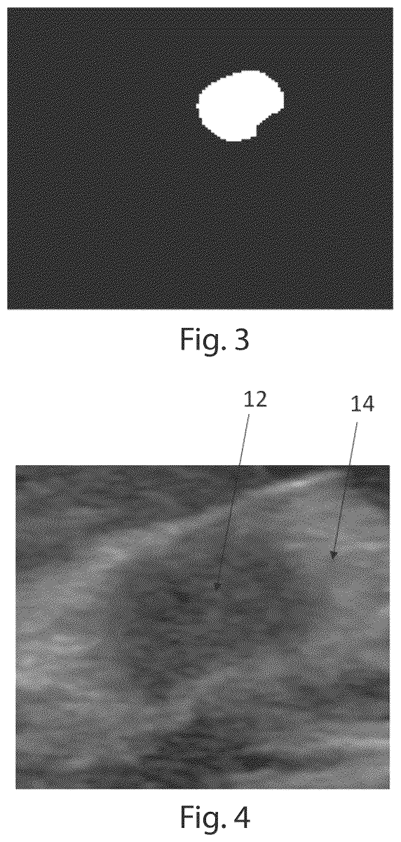

i. defining a binary mask of the same size as the input image I*, assigning an intensity value 0 to the pixels that have been designated as normal and an intensity value 1 to the pixels that have been designate as cancer (FIG. 3). The region identified by the pixels designated as cancer defines the intratumoral region.

ii. determining boundary pixels p1, p2, p3 and p4 of said intratumoral region on the binary mask.

iii. drawing a rectangle whose perimeter pixels are determined by adding a number n of proximal pixels to each boundary pixel p1, p2, p3 and p4 detected in step (ii) in order to add the intratumoral region to the peritumoral region.

iv. delineating the rectangle identified in the previous step on the input image I* to extract the ROI comprising both the intratumoral region 12 and the peritumoral region 14 (FIG. 4).

The segmentation stage 103 of the ultrasound image 1 may also comprise the extraction of a plurality of features from said ultrasound image 1.

In an embodiment, such extraction can be performed using the dec_relu4 level of the Xception architecture, which corresponds to the last activation layer of the architecture Xception before classification by means of such an architecture. In particular, the dec_relu4 level is an activation level which takes as input the convolutional layer output at the end of the network. This level has an output of size 32×32×256 corresponding to a single length vector 262144, representing the number of features extracted using the Xception architecture. Since the dec_relu4 level is one of the final levels of the Xception architecture, the extracted features corresponding to this level are features of high level, i.e., global details of the ultrasound image 1, such as, for example, tissue and morphological characteristics of the entire ultrasound image 1, which may comprise, for example, shapes and/or objects.

With reference to FIG. 1, the method 100 comprises the step of extracting 105 a plurality of characteristics of said ultrasound image 1 from said ROI using a transfer learning approach, according to which a pre-developed model for performing a given activity is used as a starting point to develop a new model that performs a different activity. The transfer learning approach is described in Pan, Sinno Jialin; Yang, Qiang (2010). “A Survey on Transfer Learning”. IEEE Transactions on Knowledge and Data Engineering, 22(10), 1345-1359. doi:10.1109/TKDE.2009.191. Such an approach allows to use a convolutional neural network previously trained on a large number of images of any type in order to be able to perform automatic extraction of features on different levels of abstraction, including both low-level features, e.g., edges, points and/or curves, and high-level features, e.g., shapes and/or objects, from an image. For example, the characteristic extraction step can be implemented by a pre-trained CNN architecture, such as the AlexNET architecture.

The extraction step 105 of a plurality of characteristics may comprise the following sub-steps:

C1. associating the indicator R with a numerical array that represents the ROI previously determined, resizing the image of the ROI to obtain an image of size 227×227 pixel, as required by the AlexNET architecture.

C2. extracting features of the re-sized ROI image from the pool2 level of the AlexNet architecture that corresponds to the second pooling level after the second convolutional layer. The pool2 level has an output of size 13×13×256 corresponding to a single vector of length 43264 units. As a consequence, the number of features extracted is 43264 in total. Since the pool2 level is one of the initial levels of the network, the extracted features corresponding to this level are low-level features, i.e., representations of local details of an image, e.g., edges, points and/or curves.

The features extracted from the ultrasound image in the segmentation stage 103, for example by using the dec_relu4 level of the Xception architecture, and the respective ROI in the extraction step 105 is aggregated in a single vector comprising said characteristics. Such an aggregation comprises juxtapositioning in said vector said one or more features extracted in step 105 to said further plurality of features extracted in step 103 from said entire ultrasound image 1.

The method 100 further comprises the step of selecting 106 the one or more characteristics from the plurality of characteristics of the image. The selection step 106 comprises selecting a plurality of features stored in said vector, wherein said plurality of selected features comprises one or more characteristics selected from said plurality of characteristics extracted from said ROI and one or more features selected from said further plurality of features extracted from said entire ultrasound image. The selection step 106 of said one or more characteristics may comprise performing one or more validation cycles to estimate a predictive power score for each of said plurality of characteristics of said image and/or said additional plurality of features extracted in step 103, wherein performing one or more validation cycles may comprise, for each cycle, performing the steps of:
a) for each of the plurality of characteristics/features, calculate an area, AUC, below a receiver operational characteristic curve, ROC, associated with said characteristic of said image and estimate said predictive power score of said image characteristic based on said AUC;
b) for each of the plurality of features, comparing said predictive power score with a selected threshold value, and in response to a determination that the predictive power score reaches said selected value, associating the image characteristic to said one or more selected features.

The selection step 106 allows to reduce the size of the set of original characteristics comprising the plurality of features extracted in step 105 of the method by identifying one or more relevant characteristics for estimating the status of the sentinel lymph node. These features are more correlated with the metastatic nodal status. Through the use of these characteristics it is possible to more accurately determine whether the primary tumor 10 has acquired the ability to migrate to the sentinel lymph node, or that has migrated, thus obtaining a more accurate estimate of the metastatic status of the sentinel lymph node.

The selection step 106 makes it possible to further increase the predictive power of the algorithm by selecting the most critical variables and eliminating those redundant and irrelevant variables for the estimation of the metastatic status.

The implementation of a selection process also makes it possible to reduce the overfitting. Moreover, the presence of one or more non-redundant features implies smaller possibilities that the noise influences the final predictions.

The selection step 106 may be performed in a Matlab® environment and may include the following sub-steps:

E1. implementing 5 cycles of an exhaustive one-layered random sampling procedure in 15 partitions to evaluate the discriminant power of each feature in predicting the status of the sentinel lymph node in the ultrasound images using the selected sub-group of images of the selected partitions. For this purpose, the following procedure may be implemented for each cycle:

i. splitting randomly and in a layered manner relative to the variable to predict the set of images in 15 partitions such that, in turn, a partition is excluded and said AUC is calculated using the images of the remaining 14 partitions, so that each image is used 14 times to test the predictive power of the single feature.

The layered aspect of the sampling enables to divide the set of images into homogeneous subpopulations with respect to the variable to be predicted, thus the percentage of patients with the metastatic lymph node is met in each partition.

The exhaustive aspect of the sampling enables the entire dataset of ultrasound images relative to the patients to be used, thus each partition is a good representing the dataset.

ii. for each cycle, computing the Area Under the Curve (AUC) of the Receiver Operating Characteristic (ROC) curve associated with each individual feature, utilizing the Matlab® perfcurve function.

The ROC curve is a graph showing the performance that a classification model reaches in assigning a positive or negative label to each image, as the threshold varies. Thus, the AUC provides an aggregated measure of performance for all the considered classification thresholds.

iii. for each cycle, selecting only features associated with an AUC value greater than or equal to a threshold value of 70%.

E2. defining the new feature set comprising one or more features selected at least 70% of the times in the 5 cycles.

With reference to FIG. 1, the method 100 further comprises performing the classification step 107 of said status of the sentinel lymph node based on one or more characteristics of said plurality of characteristics of the image selected in step 106.

The classification step 107 is performed on the basis of the features stored within the vector, and therefore it is based on the features extracted in step 105 and of the features extracted from the entire ultrasound image during the segmentation step 103. This classification allows for a more exhaustive evaluation of the metastatic status of a sentinel lymph node.

The classification step 107 comprises:
training a machine learning model to predict the status of the sentinel lymph node based on the one or more selected characteristics; the model returns a probability of positivity of the status of the sentinel lymph node.
comparing said probability with a corresponding second threshold value to estimate the metastatic status of the sentinel lymph node. The metastatic status may comprise a positive metastatic status or a negative metastatic status of said sentinel lymph node.

The classification step 107 makes it possible to discriminate between patients characterized by a positive metastatic status of the lymph node and patients whose status of the sentinel lymph node is found to be non-metastatic, and thus negative, by assigning the positive/negative label to each of the images examined. The assessment of the metastatic status of the sentinel lymph node, which is the closest to the primary tumor and the first to be reached by the tumor cells that could be migrated from the primary tumor, is an explanatory and exhaustive evaluation of the metastatic status of the surrounding lymph nodes. The real metastatic status of the sentinel lymph node of the analyzed patients was retrospectively obtained as the result of the biopsy of the sentinel lymph node, which is performed to patients with breast cancer and clinically negative lymph nodes according to the clinical practice.

To this purpose, a Support Vector Machine (SVM) classifier may be used. The SVM classifier is a supervised machine learning model that identifies, among all possible hyperplanes separating two classes of data points, the one that has the maximum margin, i.e., the maximum distance between the data points from both classes, by means of a kernel function. The classification step 107 that uses the SVM classifier may comprise the following sub-steps:

F1. training the SVM classifier by means of the Matlab® function, specifying the following input parameters: ‘Standardize’ as ‘true’, ‘KernelFunction’ as ‘linear’, ‘Kernelscale’ as ‘auto’. Thus, estimating the likelihood that an observation is classified as negative or positive by using thefitPosterior function in Matlab®.

Classify the observations using the predict function in Matlab® that calculates the probability that an observation belongs to a selected reference class.

F2. The estimated probabilities for each image are then compared with a predefined threshold value. The threshold value may be preset as the ratio between the number of images of the sentinel lymph node with positive metastatic status and the total number of available images. The images associated with a probability higher than the threshold value are assigned to the positive class. On the contrary, images characterized by a probability of less than or equal to the threshold value are assigned to the negative class.

The above-described method 100 allows to obtain a diagnostic accuracy level that is sufficient to support radiologists when evaluating the status of the sentinel lymph node.

The above-described method 100 was evaluated during validation phase, by testing the predictive ability of the method 100 on further images. The validation process may include the following sub-steps:

G1. training the SVM classifier by using of the Matlab® functionfitcsvm by considering all the images in the training set and specifying the following input parameters: ‘Standardize’ as ‘true’, ‘KernelFunction’ as ‘linear’, ‘Kernelscale’ as ‘auto’. Estimating the probability that an observation is classified as negative or positive by using thefitPosterior function of Matlab®.

G2. classifying all observations in the test set using the Matlab® function predict that calculates the probability that an observation belongs to a class.

G3. comparing the estimated probabilities for each test set image with the threshold value used in the classification step 107.

G4. evaluating the performance of the designed model in terms of Area Under the Curve (AUC) of the Receiver Operating Characteristic (ROC) curve and other standard metrics such as accuracy, sensitivity and specificity defined as follows:

$$SENSITIVITY = \frac{TP}{TP + FN} SPECIFICITY = \frac{TN}{TN + FP}$$

$$ACCURACY = \frac{TP + TN}{TN + FP + TP + FN}$$

Whereby the variable TP indicates the number of patients correctly classified as positive, the TN variable indicates the number of patients correctly classified as negative, the variable FP indicates the number of negative patients erroneously classified as positive and the variable FN indicates the number of positive patients erroneously classified as negative.

The method described above allows to obtain performance with accuracy of 90.6%, sensitivity equal to 100% and specificity equal to 88.5%.

The method described above may be performed by one or more processors that have the task of coordinating the actions needed for the execution of one or more machine instruction relating to one or more processes, passages, or steps previously described.

In some embodiments, the one or more processors may be configured to generate control information, e.g., a report related to the estimated status of the sentinel lymph node, or respond to the received information, e.g., control information from a network node.

Said one or more processors may be part of a processing system configured to classify a status of a sentinel lymph node that may also include other auxiliary components, such as a random access memory (RAM) and a read-only memory (ROM). The processing system may include a memory, such as a storage medium readable by the one or more processors.

Examples of memory include volatile and nonvolatile memory, magnetic media, optical media, random access memory (RAM), read-only memory (ROM), removable media and others. The memory may be a computer program product comprising one or more instructions that when executed by one or more processors, causes a system to perform processes, passages, or steps of the method previously described.

Advantageously, by means of the method, object of the present invention, it is possible to predict the status of a sentinel lymph node of patients with breast carcinoma in a more accurate manner by exploiting only the characteristics of various nature automatically extracted from the primary tumor ultrasound images acquired for example during the diagnosis phase.

The features extracted from the entire ultrasound image in the segmentation step 103 and those extracted in step 105 represent complementary information, which allow for a more exhaustive evaluation of the metastatic status of a sentinel lymph node. The features extracted from the entire ultrasound image in the segmentation stage 103 therefore include additional information that does not overlap with those extracted from the ROI, since they describe features of the whole image and therefore can be juxtaposed for the purposes of classification without creating redundancy. Furthermore, the depth and sensitivity levels with which such features are identified and extracted by the method described above allow to define more accurate predictive models which, importantly, are not dependent on the operators. In fact, the method according to the present invention represents a fully automated instrument starting from the acquisition of ultrasound images.

A second advantage of the method subject of the present invention is to predict the status of a sentinel lymph node of patients affected by breast carcinoma in a more robust manner since the method is based on machine learning and deep learning techniques. Such neural networks are capable of more accurately segmenting the image by properly localizing the intratumoral region 12 of the primary tumor 10 and detecting textural and morphological characteristics of the entire ultrasound image 1 and details of the ROI which are not easily identifiable by an operator thus providing a more efficient solution.

Therefore, the method described above is an accurate and non-invasive approach for the prediction of the lymphatic status in patients affected by breast cancer with clinically negative lymph nodes.

A further advantage is that the described method represents a less invasive and expensive technique with respect to other diagnostic tools. Therefore, the disclosed invention could support radiologists when estimating the status of the sentinel lymph node in a simple and accurate manner.

The present invention has been described for illustrative but not limitative purposes, according to its preferred embodiments, but it is appreciated that variations and/or modifications may be made by those skilled in the art without departing from the relative scope of protection, as defined by the appended claims.

1-15. (canceled)

16. A computer-implemented method at a processor for estimating a metastatic status of a sentinel lymph node based on an ultrasound image of a primary tumor, the method comprising:
segmenting said ultrasound image, wherein segmenting said ultrasound image includes determining a region of interest, ROI, comprising an intratumoral region of the primary tumor and a peritumoral region of the primary tumor, and wherein segmenting said ultrasound image comprises:
performing a semantic segmentation process using a first convolutional neural network, CNN, that has been trained to determine said intratumoral region of the primary tumor; and
extracting a plurality of further features from said entire ultrasound image by means of and within layers of said first CNN, said further features being high-level features, preferably wherein said further features comprise at least one of a shape and an object;
extracting a plurality of characteristics of said image from said ROI using a transfer learning approach employing a pre-trained CNN that is different than said first CNN;
aggregating into a vector said plurality of characteristics extracted from said ROI and said further plurality of features extracted from said entire ultrasound image;
selecting a plurality of characteristics stored in said vector, wherein said plurality of selected features comprises one or more characteristics selected from said plurality of characteristics extracted from said ROI and one or more features selected from said further plurality of features extracted from said entire ultrasound image; and
classifying the metastatic status of said sentinel lymph node based on said plurality of characteristic selected from said vector.

17. The method according to claim 16, comprising:
determining that the ultrasound image includes one or more markers, wherein each of said one or more markers is associated with one or more pixels of the ultrasound image; and
for each of said one or more markers, removing the marker and reconstructing said one or more pixels before segmenting said ultrasound image.

18. The method of claim 17, wherein reconstructing said one or more pixels includes performing an inpainting procedure.

19. The method of claim 16, wherein determining a ROI comprises delineating said intratumoral region of the primary tumor and said peritumor region of the primary tumor.

20. The method of claim 16, wherein extracting a plurality of characteristics of said image from said ROI includes extracting image features including low-level features, wherein said low-level features comprise representations of local details of said ROI.

21. The method of claim 20, wherein said low-level characteristics include at least one of an edge, a point and a curve.

22. The method of claim 16, wherein extracting a plurality of characteristics of said image from said ROI comprises extracting image features including high-level features, preferably wherein said high-level features include at least one of a shape and an object.

23. The method of claim 16, wherein selecting said one or more characteristics includes:
performing one or more evaluation cycles to estimate a predictive power score for each of said plurality of characteristics of said image; and
for each of said plurality of characteristics, comparing said predictive power score with a value selected from a plurality of threshold values, and in response to a determination that the predictive power score reaches the selected value, associating the image characteristic with said one or more characteristics.

24. The method of claim 23, wherein performing one or more evaluation cycles includes, for each cycle and for each of said pluralities of threshold values:
for each of said pluralities of characteristics, calculating an area, AUC, below a receiver operational characteristic curve, ROC, associated with said characteristic of said image and estimate said predictive power score of said image characteristic based on said AUC.

25. The method of claim 16, wherein classifying the metastatic status of said sentinel lymph node according to said one or more characteristics comprises:
obtaining a probability that said sentinel lymph node corresponds to a metastatic status based on said one or more selected features; and
comparing said probability with a corresponding second threshold value to estimate the metastatic status of the sentinel lymph node.

26. The method of claim 25, wherein said metastatic status is a positive metastatic status or a negative metastatic status of said sentinel lymph node.

27. A system configured to classify a metastatic status of a sentinel lymph node, the system comprising:
a memory; and
one or more processors configured to execute the method of claim 16.

28. A non-transitory computer readable medium storing one or more instructions that when executed by one or more processors, causes the one or more processors to execute the method of claim 16.
